(19) United States
(12) Patent Application Publication
(10) Pub. No.: US 2009/0260639 A1
(43) Pub. Date: Oct. 22, 2009

(54) Prevention and Treatment of Pressure Sores Using Inflatable Devices

(76) Inventors: Charles Hsu, Palo Alto, CA (US); Chienlan Hsu, Palo Alto, CA (US); Kenton Fong, Mountain View, CA (US)

Correspondence Address: MORGAN, LEWIS & BOCKIUS, LLP. (PA), 2 PALO ALTO SQUARE, 3000 EL CAMINO REAL, SUITE 700, PALO ALTO, CA, 94306, US

(21) Appl. No.: 12/427,679

(22) Filed: Apr. 21, 2009

Related U.S. Application Data
(60) Provisional application No. 61/125,008, filed on Apr. 22, 2008.

(51) Int. Cl. A61F 13/00

(52) U.S. Cl. 128/888; 602/60

(57) ABSTRACT

An inflatable device to be positioned between a body portion and an underlying surface includes one or more inflatable cells. In some embodiments, the inflatable device also includes a dressing, attached to the one or more inflatable cells, to be secured to a patient's skin. In some embodiments, the inflatable device also includes an adhesive to secure the one or more inflatable cells to a patient's skin. The inflatable device is secured to the body portion, coupled to a pump, and repeatedly inflated and deflated.

### RELATED APPLICATIONS

This application claims priority to U.S. Provisional Patent Application No. 61/125,008, titled “A Device for the Prevention and Treatment of Pressure Sores,” filed Apr. 22, 2008, which is incorporated by reference herein in its entirety.

### TECHNICAL FIELD

The disclosed embodiments relate generally to treating and preventing pressure sores, and more particularly, to inflatable devices for treating and preventing pressure sores.

### BACKGROUND

Pressure sores, also referred to as bed sores, pressure ulcers, or decubitus ulcers, are a major health care problem. These sores arise in general acute care, long-term care, and home care populations. Minimally mobile patients (e.g., ICU, spinal cord injury, elderly, and terminally ill patient populations) have the highest risk for developing pressure sores.

The development of pressure sores is primarily due to decreased blood flow to the tissues over areas of bony prominences on the body. For supine patients, sores can develop over the sacrum, heels, and back of the head. For sitting patients, sores can develop over the ischial tuberosities. For patients turned on their sides, sores can develop over the greater trochanters, hips, ankles, and knees. In any position, pressure sores can develop in any areas where there is prolonged pressure that interferes with normal blood flow into the tissue.

Intermittent pressure relief can prevent pressure sores from occurring. For example, patients who are unable to reposition themselves are sometimes required to be repositioned every two hours by nursing staff, in order to shift the pressure points. Although effective, manually repositioning patients is labor intensive and may cause back injuries in medical staff. Many institutions that do not have sufficient staff to perform repositioning in a timely fashion. Specialized mattresses and cushions can provide some relief, but also have limitations in terms of efficacy, cost, and workflow. Complicated techniques of achieving intermittent pressure relief may be unfeasible for the workflow of normal nursing care. Also, devices for achieving intermittent pressure relief should not interfere with patient access during nursing care.

### SUMMARY

In one aspect, an inflatable device to be positioned between a body portion and an underlying surface includes one or more inflatable cells. In some embodiments, the inflatable device also includes a dressing, attached to the one or more inflatable cells, to be secured to a patient's skin. In some embodiments, the inflatable device also includes an adhesive to secure the one or more inflatable cells to a patient's skin. In some embodiments, the adhesive secures a dressing to the patient's skin.

In another aspect, a system to provide pressure relief to a portion of a patient's body includes an inflatable component to be positioned between a body portion and an underlying surface; tubing coupled to the one or more inflatable cells; and a pump, coupled to the tubing, to inflate the one or more inflatable cells. The inflatable component includes one or more inflatable cells. In some embodiments, the inflatable component also includes a dressing, attached to the one or more inflatable cells, to be secured to a patient's skin. In some embodiments, the inflatable component also includes an adhesive to secure the one or more inflatable cells to a patient's skin. In some embodiments, the adhesive secures a dressing to the patient's skin.

In another aspect, a method of providing pressure relief for a body part includes securing an inflatable device to a body portion of a patient. The inflatable device includes one or more inflatable cells. In some embodiments, the inflatable device also includes a dressing attached to the one or more inflatable cells, and securing the inflatable device to the body portion includes placing the dressing on the patient's skin. In some embodiments, the inflatable device also includes an adhesive, and securing the inflatable device to the body portion includes attaching the adhesive to the patient's skin. In some embodiments, the adhesive secures a dressing to the patient's skin. The method further includes coupling the inflatable device to a pump and repeatedly inflating and deflating the inflatable device while it is secured to the body portion.

In another aspect, an inflatable device to provide pressure relief to a body part includes one or more inflatable cells, a bottom elastic surface to be positioned against an underlying support and configured to expand when the one or more inflatable cells are inflated and to contract when the one or more inflatable cells are deflated, and a top inelastic surface to be secured to a portion of a patient's body.

In another aspect, a method of providing pressure relief to a body part includes securing an inflatable device to a body portion of a patient. The inflatable device includes one or more inflatable cells, a bottom elastic surface, and a top inelastic surface. Securing the inflatable device to the body portion includes positioning the top inelastic surface under the body portion. The method further includes coupling the inflatable device to a pump and repeatedly inflating and deflating the inflatable device while the top inelastic surface remains positioned under the body portion.

In another aspect, a system to provide pressure relief to a portion of a patient's body includes an inflatable device to be positioned between a body portion and an underlying surface, tubing coupled to the one or more inflatable cells; and a pump, coupled to the tubing, to inflate the one or more inflatable cells. The inflatable device includes one or more inflatable cells, a bottom elastic surface to be positioned against the underlying surface and configured to expand when the one or more inflatable cells are inflated and to contract when the one or more inflatable cells are deflated, and a top inelastic surface to be secured to the body portion.

### BRIEF DESCRIPTION OF THE DRAWINGS

FIG. 1 is a back view of a patient showing examples of placement of inflatable components in accordance with some embodiments.

FIG. 2 is a side view of an inflatable component positioned on the posterior aspect of a patient's calf in accordance with some embodiments.

FIGS. 3A and 3B are side views illustrating inflatable components secured to a patient in accordance with some embodiments.

FIGS. 4A-4B are block diagrams illustrating systems in which a source component provides inflation to both an inflatable component and a sequential compression device in accordance with some embodiments.

FIGS. 5A-5H are plan views of examples of shapes of inflatable components in accordance with some embodiments.

FIGS. 6A-6D illustrate an inflatable component with an inelastic base in accordance with some embodiments.

FIGS. 7A and 7B are block diagrams illustrating systems in which tubing couples an inflatable component to both a source component and a suction component in accordance with some embodiments.

FIG. 8 illustrates tubing for coupling the inflatable component to a source component and/or a suction component in accordance with some embodiments.

FIGS. 9A and 9B are block diagrams illustrating systems in which a source component provides inflation to multiple inflatable components in accordance with some embodiments.

FIG. 10A illustrates a bottom view of an inflatable component positioned using a marker in accordance with some embodiments.

FIG. 10B illustrates a bottom view of an inflatable component with a clear, removable sheet having a marking in accordance with some embodiments.

FIGS. 11A-11D illustrates side views of systems that include a sequential compression device in accordance with some embodiments.

FIG. 12 is a side view illustrating an inflatable component with a dressing detachably connected to an inelastic base in accordance with some embodiments.

FIGS. 13A-13B are flow diagrams illustrating methods of providing pressure relief for a body part in accordance with some embodiments.

FIGS. 14A and 14B are side views illustrating an inflatable component positioned on a patient in accordance with some embodiments.

FIGS. 15A and 15B are plan views illustrating an “H”-shaped inflatable component and a horseshoe-shaped inflatable component in accordance with some embodiments.

Like reference numerals refer to corresponding parts throughout the drawings.

### DESCRIPTION OF EMBODIMENTS

Reference will now be made in detail to embodiments, examples of which are illustrated in the accompanying drawings. In the following detailed description, numerous specific details are set forth in order to provide a thorough understanding of the present invention. However, it will be apparent to one of ordinary skill in the art that the present invention may be practiced without these specific details. In other instances, well-known methods, procedures, components, and circuits have not been described in detail so as not to unnecessarily obscure aspects of the embodiments.

In some embodiments, an inflatable component allows for intermittent relief of pressure over specific areas of the body that are at high risk for pressure sore development. The device thus helps to prevent or reduce the development of pressure sores and treats existing pressure sores by relieving pressure such that the sores can better heal. In some embodiments, the device is designed to be localized to specific high-risk areas of the body to be minimally obtrusive to patient access, allowing for easy nursing care. The inflatable component is positioned between the patient and the bed, chair or other surface supporting the patient and is secured to the patient close to, but not directly on, areas of the body at high risk for pressure sore formation. The inflatable component is attached via tubing to a source component (e.g., an air pump or water pump) that provides intermittent inflation. The intermittent inflation and deflation provided by the source component allow the inflatable component repeatedly to elevate the high-risk area off of the underlying surface, without exerting direct pressure on the high-risk area. The inflatable component thus intermittently relieves pressure to the high-risk area, allowing perfusion and decreasing the risk of pressure sore development.

Examples of high-risk areas that the inflatable component is designed to accommodate include but are not limited to: sacrum, heels, ischial tuberosities, iliac spines, greater trochanters, scapulae, and occiput. The inflatable component may be used for areas of the body at high risk depending on patient positioning. For example, in the prone position high-risk areas include but are not limited to: sternum, rib cage, knees, toes, or shoulders. As another example, in the lateral decubitus position, high-risk areas include but are not limited to: ankles, knees, greater trochanters, shoulders, and ears.

In some embodiments, the inflatable component has a single set of one or more inflatable cells that are all inflated and deflated in synchrony, thus providing a simple design that avoids the complexity of two or more sets of inflatable components that are inflated and deflated in an alternating or otherwise asynchronous manner. Alternately, the inflatable component has two or more sets of inflatable components that may be individually inflated and deflated. For example, in some embodiments the inflatable component includes first and second independently inflatable cells. The first cell is inflated while the second cell is deflated to tilt the patient. In some embodiments the inflatable component includes multiple independently inflatable cells, and the number of cells that are inflated is varied to provide varying degrees of pressure relief or control over patient positioning. For example, additional cells could be inflated (e.g., as a function of pressure) to provide additional force to elevate a body portion of an obese patient as compared to a lighter patient. In other words, fewer cells are used to elevate a body portion of a light-weight patient as opposed to an obese patient.

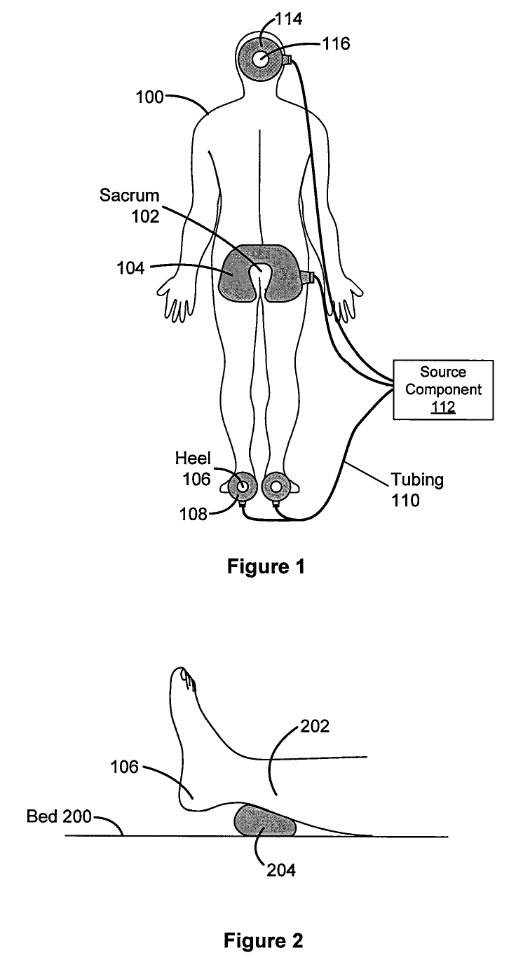

FIG. 1 is a back view of a patient 100 showing examples of placement of inflatable components 104, 108, and 114 in accordance with some embodiments. The inflatable components 104, 108, and 114 are positioned close to (e.g., around), but not directly on, areas of the body at high-risk for pressure sore development. For example, the inflatable component 104 is positioned around the patient's sacrum 102 to prevent or treat sacral pressure sores. The inflatable component 108 is positioned around the patient's heel 106 to prevent or treat heel sores. The inflatable component 114 is positioned around the back of the patient's head 116 to prevent or treat sores on the back of the head 116. In another example, as illustrated in FIG. 2 in accordance with some embodiments, an inflatable component 204 is positioned on the posterior aspect of the patient's calf or ankle 202, such that inflation of the component 204 will elevate the heel area 106 off of an underlying surface such as the bed 200 and thus help to prevent or treat pressures sores on the heel 106.

In some embodiments, positioning an inflatable component 104, 108, or 114 includes placing the inflatable component 104, 108, or 114 beneath the patient 100, such that the inflatable component 104, 108, or 114 is situated between the patient 100 and an underlying surface.

In some embodiments, the inflatable component is secured to the patient to ensure effective pressure relief. Securing the inflatable component allows for movement of the patient without misalignment of the inflatable component and may allow the inflatable component to have a small profile and overall size. Also, shear forces have been implicated in the development of pressure sores, particularly in the elderly whose skin is fragile. Securing the inflatable component to the patient may prevent shear forces (e.g., by preventing rubbing) that might otherwise also contribute to pressure sore development.

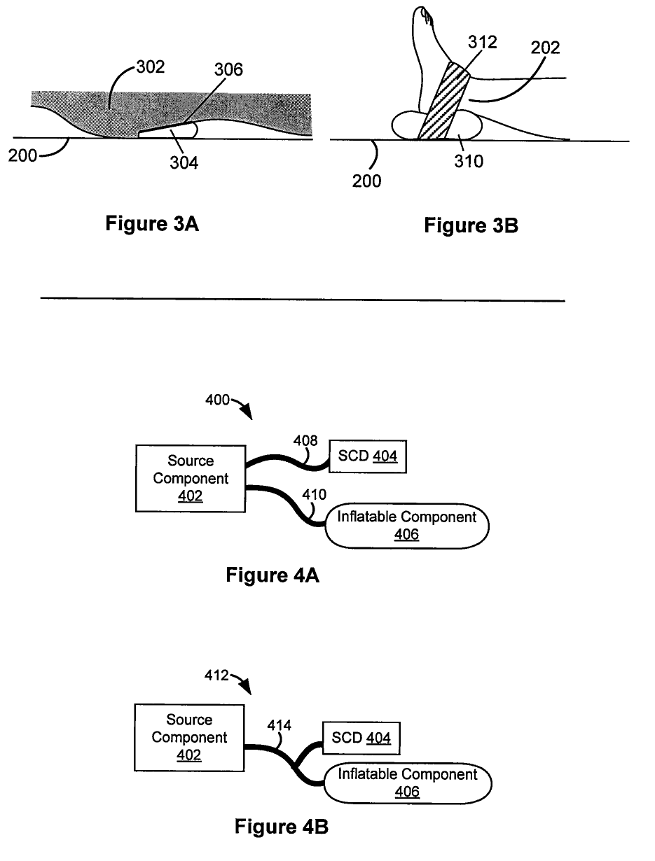

FIGS. 3A and 3B are side views illustrating inflatable components 304 (FIG. 3A) and 310 (FIG. 3B) secured to a patient in accordance with some embodiments. In FIG. 3A, an inflatable component 304 is positioned between a patient's skin 302 and an underlying surface such as a bed 200. The inflatable component 304 has a surface 306 that includes an adhesive that is placed on the patient's skin 302 to secure the inflatable component 304 directly to the patient's skin 302. In FIG. 3B, an inflatable component 310 is positioned between a patient's calf or ankle 202 (or other relevant anatomy) and an underlying surface such as the bed 200. A strap 312, sleeve, or sock is wrapped around the calf or ankle 202 and inflatable component 310 to secure the inflatable component 310 to the patient. In some embodiments, the strap 312 is attached to the inflatable component 310.

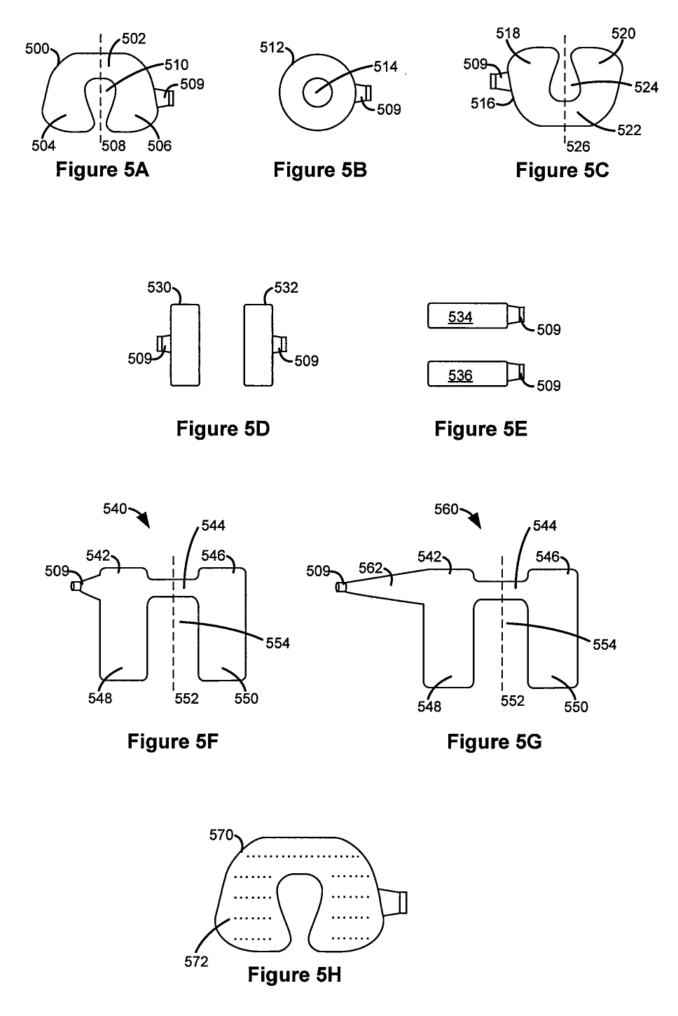

The inflatable component can take any of various shapes and sizes that enable the inflatable component to be secured close to but not immediately on an area at high risk of pressure sore formation. Any shape or size may be used, provided the contact area and shape is sufficient to at least partially relieve pressure placed on a high-risk area by the underlying bed or other surface. For example, the inflatable component may elevate the high-risk area off of the underlying bed or other surface, and may displace the bed or other surface downward (e.g., for a compressible mattress). FIGS. 5A-5H are plan views of examples of shapes of inflatable components in accordance with some embodiments. Each inflatable component includes an attachment (e.g., a plastic attachment) 509 to connect the inflatable component to tubing that couples the inflatable component to a source component (e.g., a pump) that provides inflation. In some embodiments, as illustrated in FIG. 5B, an inflatable component 502 has a circular shape with an open or depressed center 514 to accommodate the high-risk area. In some embodiments, as illustrated in FIGS. 5A and 5C, inflatable components 500 and 516 have a “U” shape (e.g., a horseshoe shape) with an opening 510 or 524 to accommodate the high-risk area. In some embodiments, as illustrated in FIGS. 5F and 5G, an inflatable component 540 or 560 has an “H” shape. In some embodiments, the “H”-shaped inflatable component 560 has an extension 562 configured to ensure that the attachment 509 is not underneath the patient. In some embodiments, the inflatable components 500, 512, 516, 540 and 560 thus are configured to at least partially surround but not to cover a portion 510, 514, 524, or 554 of the patient's skin. In some embodiments, as illustrated in FIGS. 5D and 5E, inflatable components 530, 532, 534, and 536 have a bar shape. Bar-shaped inflatable components may be placed on opposite sides of a body portion to elevate the portion. In other examples, the inflatable component has a crescent shape or another anatomically configured shape depending on the location of the pressure sore, lesion, or high-risk area. Inflatable components such as the components 500, 512, 516, 530-536, 540 and 560 thus are anatomically designed to fit the contours of the surrounding tissues of areas where pressure sores commonly occur.

The U-shaped inflatable components 500 and 516 (FIGS. 5A and 5C) have a central portion 502/524 and a pair of protruding portions (504 and 506, FIG. 5A, or 518 and 520, FIG. 5C) extending outward from the central portion 502/524 on both sides of a longitudinal axis 508/526. Similarly, the H-shaped inflatable components 540 (FIG. 5F) and 560 (FIG. 5G) have a central portion 544, a first pair of protruding portions 548 and 550 extending outward from the central portion 544 in a first direction on both sides of a longitudinal axis 552, and a second pair of protruding portions 542 and 546 extending outward from the central portion 544 in a second direction opposite to the first direction on both sides of the longitudinal axis 552. The “H” shape helps to ensure that the portions 548 and 550 do not round off when inflated such that they enclose the area 554. Leaving the area 554 open on at least one side of the inflatable component 540 or 560 improves blood flow to the area 554, thus promoting healing or prevention of pressure sores. In some embodiments, the central portion 544 of the H-shaped component 540 or 560 has a width less than or equal to a value selected to prevent excessive arching of the patient's back, where width refers to the dimension in the direction of the axis 552. For example, the central portion 544 may have a width when deflated of 5 inches or less, or 4 inches or less.

In some embodiments, an inflatable component includes a plurality of holes (e.g., pinholes) in the surface to be positioned against the patient's skin. The holes allow air to escape from the inflatable component when inflated and thus provide airflow through the surface that serves to dry the skin and prevent excessive moisture from macerating the skin. FIG. 5H illustrates an inflatable device 570 having a plurality of such holes 572 in accordance with some embodiments. In some embodiments, each hole 572 is less than 2 mm² in area, or less than 1 mm² in area, or less than 0.5 mm² in area.

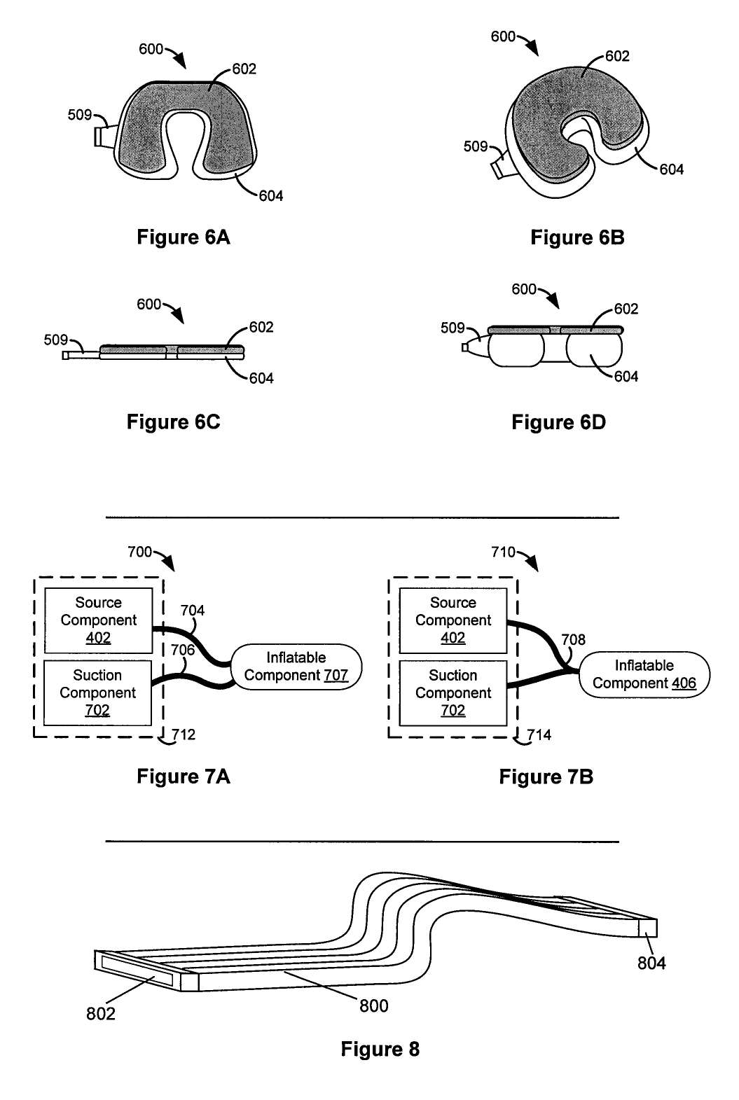

FIGS. 6A-6D illustrate an inflatable component 600 with an inelastic base 602 in accordance with some embodiments. In some embodiments, the inelastic base 602 is plastic. The inelastic base 602 may be positioned on (e.g., secured to) the patient's skin. In some embodiments, a dressing is attached to the inelastic base 602. As illustrated in FIGS. 6C-6D, the inelastic base 602 generally maintains its shape regardless of whether the component 600 is inflated (FIG. 6D) or deflated (FIG. 6C), thus avoiding shear forces on the patient's skin and helping to prevent pressure sore formation. In some embodiments, the one or more inflatable cells 604 of the component 600 are configured to flatten smoothly against the inelastic base 602 when deflated (FIG. 6C), such that there are no folds or wrinkles in the surface material of the one or more inflatable cells 604. (Folds or wrinkles can cause discomfort for the patient and contribute to pressure sore formation.) For example, the one or more inflatable cells 604 have an elastic surface that is attached to the inelastic base 602 and smoothly flattens against the inelastic base 602 when deflated. In some embodiments, instead of having an elastic surface attached to an inelastic base, the one or more inflatable cells 604 have an elastic surface on a first side and an inelastic surface on a second side; the elastic surface smoothly flattens against the inelastic surface upon deflation, while the inelastic surface holds its shape. In some embodiments, a dressing is attached to the inelastic surface.

In some embodiments, the inelastic base 602 or inelastic surface is secured to the body portion using a strap or sleeve. Alternatively, an adhesive is used to attach the inelastic base 602 or inelastic surface to a body portion of the patient.

In some embodiments, the one or more inelastic cells that constitute the inflatable component are entirely inelastic. For example, the one or more inelastic cells may be made of cloth-covered vinyl.

In some embodiments (e.g., as discussed for the inelastic base 602), the inflatable component is attached to the body through a dressing (e.g., on the surface 306, FIG. 3A) that is attached to the one or more inflatable cells of the inflatable component. For example, the dressing may be fixedly attached to one or more inflatable cells such that it is integrated into a surface of the one or more inflatable cells (e.g., the dressing is glued or sewn onto the surface or forms part of the surface and is attached to the remainder of the surface, for example by being sewn around its edges). In some embodiments, the dressing is moisture-absorbent. For example, a hydrocolloid tissue interface such as Duoderm® (a registered trademark of E.R. Squibb & Sons) is used to provide a breathable semi-permeable dressing that can absorb moisture and remain in place for extended periods of time. Because poor moisture control has been implicated in the development of pressure sores, use of a moisture absorbent and/or breathable dressing helps to prevent pressure sores. In some embodiments, the dressing is adhesive and thus is used to attach or secure the inflatable component to the body. For example, hydrocolloid dressings are adhesive. Alternatively (e.g., if the dressing is not adhesive), the dressing is secured against the body using a separate adhesive or a strap 312 (FIG. 3B), sleeve, or sock.

In some embodiments, the dressing spans an opening (e.g., 510, 514, 524, or 554, FIGS. 5A-5C and 5F-5G) on the surface of the inflatable component (e.g., an opening that the inflatable component at least partially surrounds), such that the dressing covers the high-risk area associated with the opening. In some embodiments, the dressing has dimensions of between 15-38 cm on a side.

In some embodiments, the dressing includes an absorbent material such as, for example, comminuted wood pulp (generally referred to as airfelt), creped cellulose wadding, absorbent foams, absorbent sponges, super absorbent polymers, absorbent gelling materials, or any equivalent materials or combination of materials. In some embodiments, the dressing includes an absorbent material made of cellulosic fiber, such as, for example, rayon, lyocell, wood pulp, cotton, any superabsorbent, such as, for example, polyacrylate, or some combination of these types of fibers. Alternatively, the superabsorbent may be in powder form or granular form.

In some embodiments, the dressing includes an outer layer of moisture-wicking material to be positioned against a patient's skin. In some embodiments, the dressing further includes an absorbent layer beneath (e.g., adjacent to) the outer layer of moisture-wicking material.

In some embodiments, the dressing includes skin moisturizer to moisturize the patient's skin. For example, the dressing may be infused with moisturizer or have a top layer to be placed on the patient's skin that has moisturizer in or on it.

In some embodiments, an inflatable component includes an attached dressing that is detachable from the one or more inflatable cells. For example, FIG. 12 is a side view illustrating an inflatable component 1200 with a dressing 1202 detachably connected to an inelastic base 602 in accordance with some embodiments. In some embodiments, a hook-and-loop material is used to attach the dressing 1202 to the inflatable component. For example, velcro layers 1204 and 1206 on the back side of the dressing 1202 and the inelastic base 602 couple the one or more inflatable cells 604 to the dressing 1202. In some embodiments, the dressing 1202 is detachably connected to the inelastic base 602 using an adhesive between the dressing 1202 and the inelastic base 602. The dressing 1202 may be removed and replaced with another dressing 1202, and thus is disposable. In some embodiments, the dressing 1202 is attached to the patient's skin using an adhesive.

In some embodiments, the inflatable component 1200 includes an adhesive to attach the dressing 1202 to the patient's skin. For example, the dressing 1202 itself may be adhesive (e.g., it may be a hydrocolloid material). The dressing 1202 is configured to detach from the one or more inflatable cells in response to an applied force that is less than the force required to detach the adhesive from the patient's skin, thereby ensuring that an attempt to detach the dressing 1202 from the one or more inflatable cells does not accidentally result in pulling the dressing 1202 off of the patient's skin instead. For example, the force required to separate the velcro layers 1204 and 1206 is less than the force required to pull the dressing off of the patient's skin.

In some embodiments, the inflatable component is fabricated using a waterproof and/or fire-repellent material. For example, the inflatable component may include a waterproof and/or fire-repellent cover. In some embodiments, the inflatable component includes a removable cover that can be removed and then cleaned or replaced with a new cover. The removable cover may be a dressing, such as those described above.

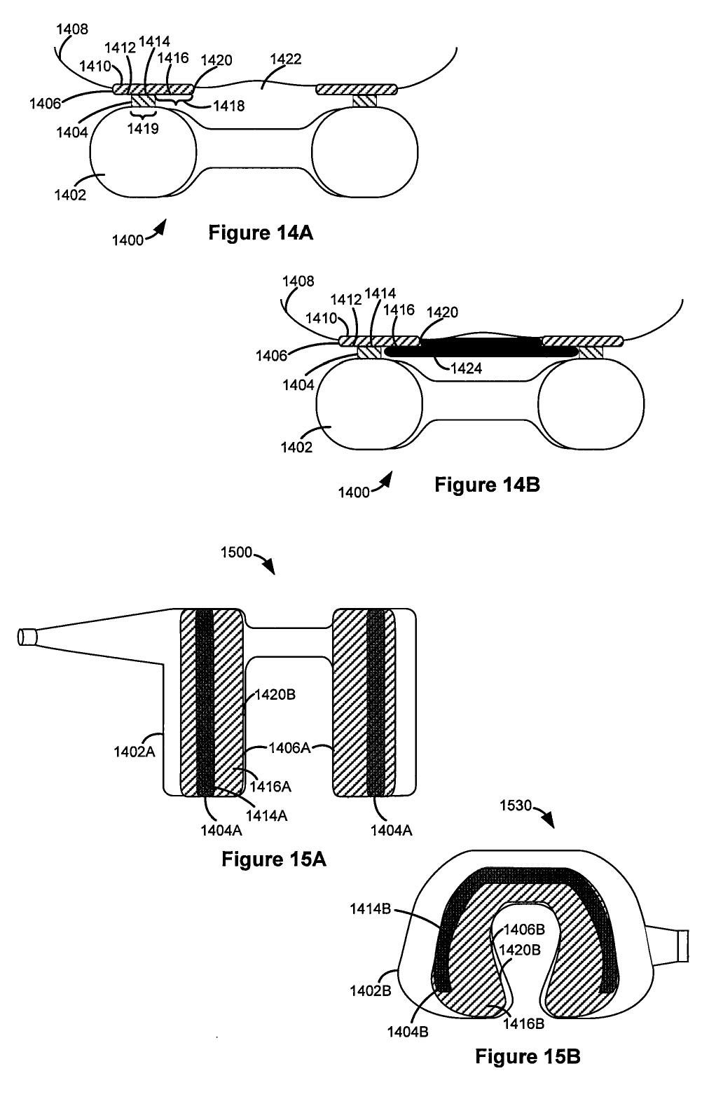

FIGS. 14A and 14B are side views illustrating an inflatable component 1400 (e.g., a horseshoe-shaped, “H”-shaped, or ring-shaped component) positioned on a patient 1408 in accordance with some embodiments. In some embodiments, the inflatable component 1400 surrounds or partially surrounds an opening 1422. The inflatable component 1400 includes a dressing 1406 connected to one or more inflatable cells 1402 by connective material 1404. In some embodiments, the dressing 1406 is detachable from the one or more inflatable cells 1402. For example, the connective material 1404 includes detachable hook-and-loop material. In some embodiments, the connective material 1404 includes an adhesive to either fixedly or detachably attach the dressing 1406 to the one or more inflatable cells 1402.

The dressing 1406 includes a first side 1410 to be positioned against the skin of the patient 1408 and a second side 1412 opposite the first side 1410. The second 1412 includes a first area 1414 in contact with the connective material 1404 and a second area 1416 between the first area 1414 and an edge 1420 of the dressing (e.g., the edge facing the at least partially enclosed area 1422) that is not in contact with the connective material 1404. The second area 1416 has a width 1418 sufficient to attach an external dressing 1424 to the inflatable component 1400, as illustrated in FIG. 14B in accordance with some embodiments. The external dressing 1424 may be attached to the inflatable component 1400 using tape applied to the second area 1416. Alternatively, the external dressing itself may be adhesive and thus may be adhesively connected to the second area 1416. The second area 1416 thus has a width 1418 sufficient to attach the tape or the external dressing 1424. In some embodiments, the external dressing 1424 spans the at least partially enclosed area 1422. In some embodiments, the width 1418 of the second area 1416 is at least 1 cm, or at least 1.5 cm, or at least 1.8 cm, or at least 2 cm. In some embodiments, the second area 1416 includes a zipper to attach the external dressing 1424 to the second area 1416. In some embodiments, the second area 1416 includes a groove of a rib-and-grove connector to attach the external dressing 1424 to the second area 1416, the rib being located on the perimeter of the external dressing 1424.

In some embodiments, the inflatable component 1400 has an elastic surface that tends to ball up when inflated. In some embodiments, the connective material 1404 has a width 1419 that substantially minimizes curling of the dressing 1406 upon inflation of the one or more cells 1402, such that the dressing 1406 remains positioned on and conformal to the skin of the patient 1408 when the one or more cells 1402 are inflated. In some embodiments, the dressing 1406 has a width of at least 2 inches, or at least 3 inches, or at least 5 inches, and the connective material has a width of no more than 1 inch, or no more than 0.8 inches, or no more than 0.5 inches.

FIGS. 15A and 15B are plan views illustrating an “H”-shaped inflatable component 1500 and a horseshoe-shaped inflatable component 1530 in accordance with some embodiments. The “H”-shaped inflatable component 1500 and horseshoe-shaped inflatable component 1530 are examples of inflatable components 1400 (FIGS. 14A-14B) and include connective material 1404A or 1404B situated between and connecting one or more inflatable cells 1402A or 1402B and a dressing or dressings 1406A or 1406B, such that a first area 1414A or 1414B is in contact with the connective material 1404A or 1404B and a second area 1416A or 1416B is between the first area 1414A or 1414B and an edge 1420A or 1420B of the dressing.

In some embodiments, the pump used to inflate and deflate the inflatable component is integrated with one of various types of pumps used in hospital or nursing care settings. For example, the pump may be integrated into a source component (e.g., an air pump) for sequential compression device (SCD) systems used to minimize the risk of deep vein thrombosis. In other words, the source component (air pump or otherwise) may be configured to be connectable to existing SCD systems, such that the source component may provide inflation to both an inflatable component (e.g., 502, 512, 516, 530-536, 540, 560, or 570, FIGS. 5A-5H) as well as existing SCDs, as illustrated in FIGS. 4A and 4B in accordance with some embodiments. In the system 400 of FIG. 4A, a source component 402 provides inflation for an SCD 404 through tubing 408 and provides inflation for an inflatable component 406 through separate tubing 410. In some embodiments, the first tubing 408 connects to a first connection outlet in the source component 402 and the second tubing 410 connects to a second connection outlet in the source component 402. Alternately, in the system 412 of FIG. 4B, the source component 402 provides inflation for the SCD 404 and inflatable component 406 through a single shared tubing 414 that connects to a single connection outlet in the source component 402 and branches to the SCD 404 and inflatable component 406. The tubing connections for the inflatable component 406 thus may be compatible with SCD pumps and an SCD pump may act as the source component 402 for the inflatable component 406.

In some embodiments, the pump is integrated into or combined with a pump for a negative-pressure wound therapy system. In some embodiments, the pump is integrated into or combined with a pump for an air mattress.

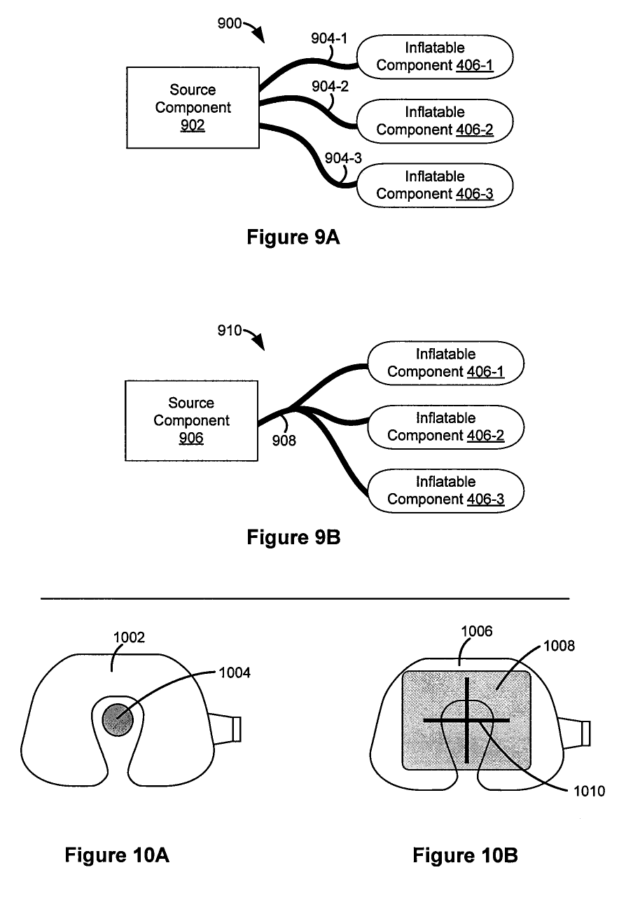

In some embodiments a source component has multiple outlets (and, in some embodiments, inlets) that allow it to be attached to multiple inflatable components to treat multiple areas of the body in a single person or to treat multiple persons. FIG. 9A illustrates a system 900 in which a source component 902 has multiple connection outlets connecting to multiple respective tubings 904-1 through 904-3 for providing inflation to multiple respective inflatable components 406-1 through 406-3, in accordance with some embodiments. In some embodiments, the source component has a single outlet (and, in some embodiments, inlet) that connects to tubing that branches and provides inflation and deflation to more than one inflatable component. FIG. 9B illustrates a system 910 in which a source component 906 has a single connection outlet for a single tubing 908 that branches to the multiple inflatable devices 406-1 through 406-3, in accordance with some embodiments.

In some embodiments, to reduce noise, the source component has a casing made of foam, fiberglass, or other sound insulating or absorbing material.

In some embodiments, the source component (e.g., 112 (FIG. 1), 402 (FIGS. 4A-4B), 902 (FIG. 9A), or 906 (FIG. 9B)) has an electronic system for controlling the intermittent inflation and deflation of an inflatable component (e.g., 406, FIGS. 4A-4B and 9A-9B). The intermittent inflation and deflation can be timed in any of various ways. The inflation and deflation rates can be rapid or slow. Examples of rapid deflation rates include 5 seconds or less, or 10 seconds or less, while examples of slow deflation rates include at least 2 minutes, or at least 5 minutes, or 10 minutes or more. In some embodiments, the inflation frequency varies from every 5 minutes to every 4 hours. Examples of inflation frequencies include 5-10 minutes, 20 minutes or less, an hour or less, 1-4 hours, or 2-4 hours. In some embodiments, the duration of inflation varies from 30 seconds to 2 hours. Examples of durations of inflation include 1 minute or less, 2 minutes or less, 10 minutes or less, 30 minutes to 2 hours, or 1 to 2 hours. A rapid rate of inflation and deflation produces rapid blood reperfusion to the high-risk area, thus aiding healing or prevention of pressure sores. A lower rate of inflation and deflation, however, is more comfortable to the patient, both because the patient is not subjected to sudden changes and because pump noise is reduced. In some embodiments, the inflation and deflation rates, inflation frequency, and/or duration of inflation are varied depending on the time of day, the condition of the patient, the weight of the patient, a degree or other measure of bed softness, whether the patient is awake or asleep, the patient's position (e.g., supine, prone, lateral decubitus, or tilted), and/or one or more other user-defined conditions. In some embodiments, the inflation and deflation rates, inflation frequency, and/or duration of inflation are programmed to constantly vary to reduce patient discomfort associated with a particular inflation or deflation rate, inflation frequency, or duration of inflation.

In some embodiments, the inflatable component deflates passively. In some other embodiments, however, a mechanical pump deflates the inflatable component. The advantage of such an active deflation is that it reduces the possibility of creating new pressure points from the inflatable component itself because the inflatable component is suctioned flat.

In some embodiments, an inflatable component has a tube or tubes that connect to a suction component to provide active deflation. FIG. 7A illustrates a system 700 in which an inflatable component 707 is coupled to a source component 402 through a first tubing 704 and a suction component 702 through a second tubing 706 in accordance with some embodiments. FIG. 7B illustrates an alternative system 710 in which a single, branching tubing 708 couples an inflatable component 406 to the source component 402 and the suction component 702 in accordance with some embodiments. The source component 402 and suction component 702 may be combined into a single component 712 (FIG. 7A) or 714 (FIG. 7B) that provides both inflation and deflation.

FIG. 8 illustrates tubing 800 for coupling the inflatable component to a source component, and in some embodiments a suction component, in accordance with some embodiments. The tubing 800, which is an example of tubing 408, 410, and/or 414 (FIGS. 4A-4B) and of tubing 704, 706, and 708 (FIGS. 7A-7B), includes a connector 802 to connect to the inflatable component and a connector 804 to connect to a source component or a suction component. In some embodiments, the tubing 800 is designed to be low-profile, such that it does not itself become a source for further pressure sores.

In some embodiments, the source component (e.g., 402, FIG. 4) provides a uniform protocol of inflation and deflation (e.g., level, frequency and/or duration of inflation) optimized to the vast majority of patients. In some embodiments, the source component has one or more pumps that can deliver different (e.g., variable) levels of inflation to the inflatable component. In some embodiments, there is a control panel, one or more switches, or one or more dials that can be used to adjust the function of the source component (e.g., to adjust the level, frequency, and/or duration of inflation). Examples of control panels include but are not limited to a simple dial or an electronic display that may take various inputs (e.g., the patient's body weight or the bed type) to determine the amount of inflation. In some embodiments, for example, the degree of inflation is adjusted via manual or electronic controls for the patient's body habitus (i.e., build), weight, or gender, as well as for characteristics of the mattress the patient is lying upon, such as the mattress type (e.g., mattress manufacturer and model), or characteristics of the chair the patient is sitting upon, such as the type of chair (e.g., manufacturer, and model). In some embodiments, the amount of inflation is determined by using a look-up table that stores inflation values corresponding to different control panel settings. Frequency and duration of inflation may be similarly determined using a look-up table. Alternatively, the level, frequency, and/or duration of inflation may be directly specified using the control panel, switches, and/or dials.

In some embodiments, the inflatable component has a pressure sensor built into it that provides feedback to the source component. In some embodiments the source component can adjust inflation pressures based on the pressure sensor readings to ensure that pressure is alleviated adequately to prevent pressure sore formation. In some embodiments, the pressure sensor is within the source component itself and may adjust to the pressure required for adequate inflation of the elevation component. In some embodiments, the source component includes or is coupled to one or more additional sensors (e.g., temperature, motion, blood oxygen, or pulse sensors).

In some embodiments, a positioning component, or marking, is used to align the inflatable component to an area of treatment (e.g., a high-risk area, or an area having a pressure sore or lesion). FIG. 10A illustrates an inflatable component 1002 positioned using a marker 1004 in accordance with some embodiments. In some embodiments, the marking 1004 is a removable sticker, separate from the inflatable component 1002, which is placed on the area of treatment to allow the inflatable component 1002 to be positioned around the area of treatment. FIG. 10B illustrates an inflatable component 1006 with a clear, removable sheet 1008 in accordance with some embodiments. The removable sheet 1008 has a marking 1010 (e.g., an “X” or cross) that can be aligned with the area of treatment to position the inflatable component 1006 around the area of treatment.

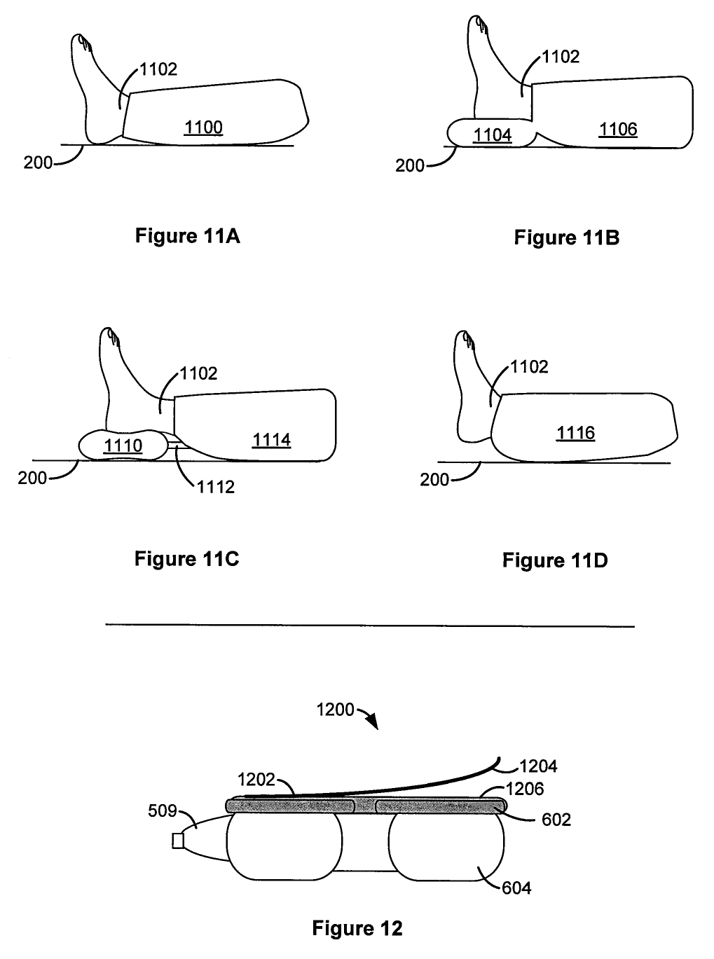

In some embodiments, the inflatable component includes a sequential compression device (SCD) as well as an inflatable extension (e.g., a distal extension) to relieve pressure from a high-risk area. FIG. 11A illustrates an SCD 1100 configured to receive a patient's extremity (e.g., a lower extremity, or leg) 1102. In FIG. 11B, by comparison, an inflatable extension 1104 is coupled to an SCD 1106 in accordance with some embodiments: the SCD 1106 receives a lower extremity 1102, and the inflatable extension 1104 extends distally beyond the SCD 1106 to relieve pressure from the heel during SCD 1106 inflation. The inflatable extension 1104 is positioned under or around the heel and elevates the heel when inflated. The inflatable extension 1104 includes one or more inflatable cells. In FIG. 11B, the inflatable extension 1104 is directly connected to the SCD 1106. Alternatively, an inflatable extension 1110 is coupled to an SCD 1114 through tubing 1112, as illustrated in FIG. 11C in accordance with some embodiments. The inflatable extension 1110, like the inflatable extension 1104, relieves pressure from the heel when inflated. In some embodiments, an inflatable component 1116 does not have a separate extension, but instead includes an SCD and is configured to provide asymmetric inflation (e.g., a greater volume of inflation underneath the extremity 1102 than above the extremity 1102) such that inflation of the component 1116 around the calf will elevate the heel off of an underlying surface such as the bed 200, as illustrated in FIG. 11D in accordance with some embodiments.

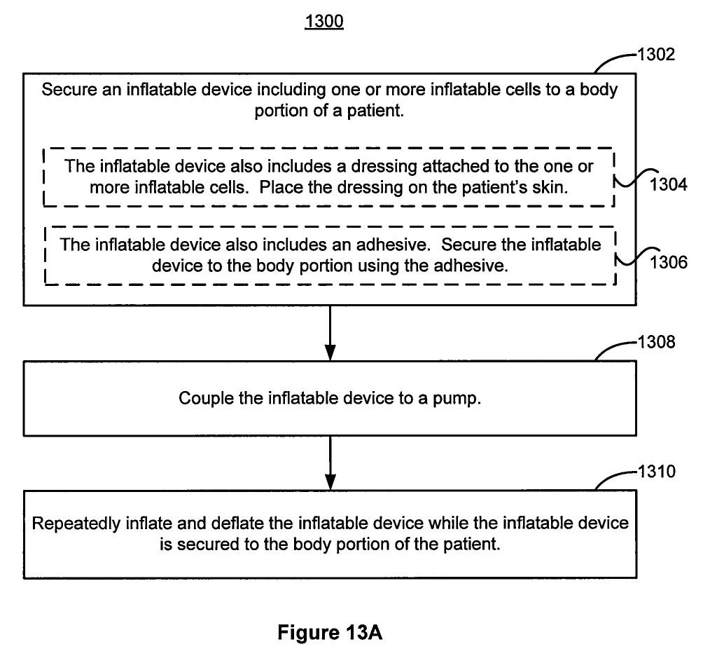

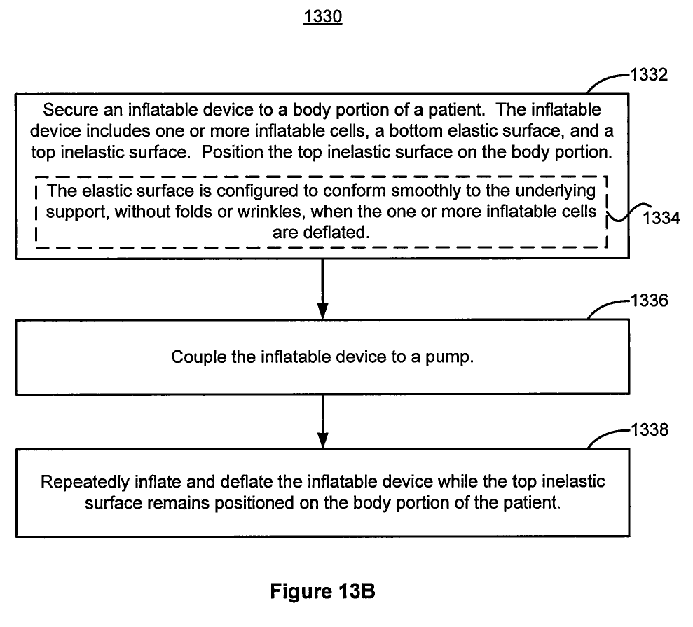

FIG. 13A is a flow diagram illustrating a method 1300 of providing pressure relief for a body part in accordance with some embodiments. In the method 1300, an inflatable device that includes one or more inflatable cells is secured (1302) to a body portion of a patient (e.g., patient 100, FIG. 1). Examples of inflatable devices include inflatable components 104 and 108 (FIG. 1); 204 (FIG. 2); 304 and 310 (FIGS. 3A-3B); 406 (FIGS. 4A-4B, 7B, and 9A-9B); 500, 512, 516, 530-536, 540, 560, and 570 (FIGS. 5A-5H); 600 (FIGS. 6A-6D); 707 (FIG. 7A); 1002 and 1006 (FIGS. 10A-10B); 1104 and 1110 (FIGS. 11B-11C); 1200 (FIG. 12); and 1400 (FIGS. 14A-14B). In some embodiments, the inflatable device also includes a dressing (e.g., a dressing on the surface 306, FIG. 3A, or the dressing 1202, FIG. 12) attached to the one or more inflatable cells, and the dressing is placed (1304) on the patient's skin. In some embodiments, the inflatable device also includes an adhesive (e.g., an adhesive on the surface 306, FIG. 3A), and the inflatable device (which may or may not also include the dressing) is secured (1306) to the body portion using the adhesive. Alternatively, in some embodiments the inflatable device is secured to the body portion using a strap (e.g., 312, FIG. 3B) or sleeve. The inflatable device is coupled (1308) to a pump (e.g., in a source component 402, FIGS. 4A-4B and 7A-7B, 902, FIG. 9A, or 906, FIG. 9B) and repeatedly inflated and deflated (1310) while the inflatable device is secured to the body portion of the patient. While the method 1300 includes a number of operations that appear to occur in a specific order, it should be apparent that the method 1300 can include more or fewer operations, the order of two or more operations may be changed, and/or two or more operations may be combined into a single operation.

FIG. 13B is a flow diagram illustrating a method 1330 of providing pressure relief for a body part in accordance with some embodiments. In the method 1330, an inflatable device (e.g., 600, FIGS. 6A-6D) that includes one or more inflatable cells (e.g., 604, FIGS. 6A-6D), a bottom elastic surface, and a top inelastic surface (e.g., inelastic base 602, FIGS. 6A-6D) is secured (1332) to a body portion of a patient. The top inelastic surface is positioned on the body portion.

In some embodiments, the elastic surface is configured to flatten smoothly (1334) against the underlying support, without folds or wrinkles, when the one or more inflatable cells are deflated (e.g., as illustrated in FIG. 6C).

The inflatable device is coupled (1336) to a pump (e.g., in a source component 112 (FIG. 1), 402 (FIGS. 4A-4B and 7A-7B), 902 (FIG. 9A), or 906 (FIG. 9B)) and repeatedly inflated and deflated (1338) while the top inelastic surface remains positioned on the body portion of the patient. The method 1330 thus provides pressure relief while avoiding shear forces on the patient's skin that could irritate the skin or contribute to pressure sore formation.

In some embodiments, the one or more inflatable cells include first and second independently inflatable cells. The first inflatable cell is inflated when the second inflatable cell is deflated, to tilt the patient (e.g., to help offload the patient from a bed or chair).

While the method 1330 includes a number of operations that appear to occur in a specific order, it should be apparent that the method 1330 can include more or fewer operations, the order of two or more operations may be changed, and/or two or more operations may be combined into a single operation.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the invention and its practical applications, to thereby enable others skilled in the art to best utilize the invention and various embodiments with various modifications as are suited to the particular use contemplated.

What is claimed is:

1. An inflatable device to be positioned between a body portion and an underlying surface, comprising:
one or more inflatable cells; and
a dressing, attached to the one or more inflatable cells, to be secured to a patient's skin.

2. The inflatable device of claim 1, wherein the dressing is integrated into a surface of the one or more inflatable cells.

3. The inflatable device of claim 1, wherein the dressing comprises a hydrocolloid material.

4. The inflatable device of claim 1, comprising an adhesive to attach the dressing to the patient's skin.

5. The inflatable device of claim 1, wherein the dressing comprises a moisture-absorbent material.

6. The inflatable device of claim 1, wherein the dressing comprises a breathable material.

7. The inflatable device of claim 1, further comprising an adhesive to secure the dressing to the patient's skin.

8. The inflatable device of claim 1, further comprising:
an attachment to couple the one or more inflatable cells to tubing for inflating and deflating the one or more inflatable cells.

9. The inflatable device of claim 1, further comprising:
a marking to be aligned to an area of treatment on the patient's skin.

10. The inflatable device of claim 1, further comprising:
a removable sheet having a marking to be aligned to an area of treatment on the patient's skin.

11. The inflatable device of claim 1, comprising:
an inelastic base attached to the one or more inflatable cells, wherein the dressing is attached to the inelastic base.

12. The inflatable device of claim 11, wherein the one or more inflatable cells comprise an elastic surface configured to flatten smoothly against the inelastic base upon deflation of the one or more inflatable cells.

13. The inflatable device of claim 1, comprising:
an inelastic material forming a first surface of the one or more inflatable cells.

14. The inflatable device of claim 13, further comprising:
an elastic material, forming a second surface of the one or more inflatable cells, configured to flatten smoothly against the inelastic first surface upon deflation of the one or more inflatable cells.

15. The inflatable device of claim 1, wherein the one or more inflatable cells are configured to elevate a specific part of the patient's body when inflated.

16. The inflatable device of claim 15, wherein the one or more inflatable cells are configured to fit contours of tissue surrounding an area at risk of pressure sore formation.

17. The inflatable device of claim 1, wherein the one or more inflatable cells have a horseshoe-shaped configuration.

18. The inflatable device of claim 1, wherein the one or more inflatable cells have an “H” shape.

19. The inflatable device of claim 1, wherein the one or more inflatable cells comprise:
a central portion; and
a first pair of protruding portions extending outward from the central portion in a first direction on both sides of a longitudinal axis; and

20. The inflatable device of claim 19, wherein the one or more inflatable cells further comprise:
a second pair of protruding portions extending outward from the central portion in a second direction opposite to the first direction on both sides of the longitudinal axis.

21. The inflatable device of claim 20, wherein the central portion has a width of 5 inches or less when deflated.

22. The inflatable device of claim 1, wherein the one or more inflatable cells have a crescent-shaped configuration.

23. The inflatable device of claim 1, further comprising:
a sequential compression device coupled to the one or more inflatable cells, the one or more inflatable cells configured to extend outward from the sequential compression device.

24. The inflatable device of claim 23, wherein:
the sequential compression device is configured to receive the patient's leg; and
the one or more inflatable cells are configured to extend distally beyond the sequential compression device to elevate the patient's heel when inflated.

25. The inflatable device of claim 1, wherein:
the one or more inflatable cells are configured when inflated to at least partially surround a portion of the patient's skin but not to cover the portion of the patient's skin that is at least partially surrounded.

26. The inflatable device of claim 25, wherein:
the dressing is configured to cover the portion of the patient's skin that is at least partially surrounded.

27. The inflatable device of claim 1, wherein the dressing is detachable from the one or more inflatable cells.

28. The inflatable device of claim 27, comprising hook-and-loop material to detachably couple the one or more inflatable cells to the dressing.

29. The inflatable device of claim 27, wherein the dressing is disposable.

30. The inflatable device of claim 27, comprising an adhesive to attach the dressing to the patient's skin, wherein the dressing is configured to detach from the one or more inflatable cells in response to an applied force less than a force required to detach the adhesive from the patient's skin.

31. The inflatable device of claim 30, wherein the dressing includes the adhesive.

32. The inflatable device of claim 1, wherein the dressing comprises skin moisturizer to moisturize the patient's skin.

33. The inflatable device of claim 1, wherein the dressing comprises:
an outer layer of moisture-wicking material.

34. The inflatable device of claim 33, wherein the dressing further comprises:
an absorbent layer adjacent to the outer layer of moisture-wicking material.

35. The inflatable device of claim 1, wherein a surface of the one or more inflatable cells includes a plurality of holes to provide airflow through the surface when the one or more inflatable cells are inflated.

36. The inflatable device of claim 1, the dressing having a first side to be positioned against the patient's skin and a second side opposite to the first side, the inflatable device further comprising:
a connective material to connect the one or more inflatable cells to the second side of the dressing;
wherein the second side of the dressing comprises a first area in contact with the connective material and a second area, between the first area and an edge of the dressing, that is not in contact with the connective material.

37. The inflatable device of claim 36, wherein the second area has a width between the first area and the edge of the dressing of at least 1 cm.

38. The inflatable device of claim 36, wherein the second area has a width between the first area and the edge of the dressing of 2 to 4 cm.

39. The inflatable device of claim 36, wherein the second area has sufficient width to attach an external dressing to the inflatable device.

40. The inflatable device of claim 36, wherein the second area includes a zipper to attach an external dressing to the inflatable device.

41. The inflatable device of claim 36, wherein the second area includes a groove of a rib-and-grove connector to attach an external dressing to the inflatable device.

42. The inflatable device of claim 36, wherein the connective material comprises hook-and-loop material.

43. The inflatable device of claim 36, wherein the connective material comprises an adhesive.

44. The inflatable device of claim 1, the dressing having a first side to be positioned against the patient's skin and a second side opposite to the first side, the one or more inflatable cells including an elastic surface, the inflatable device further comprising:
a connective material to connect the elastic surface of the one or more inflatable cells to the second side of the dressing, the connective material having a width that substantially minimizes curling the dressing upon inflation of the one or more inflatable cells.

45. The inflatable device of claim 44, wherein the dressing has a width of at least 2 inches and the connective material has a width of no more than 1 inch.

46. A system to provide pressure relief to a portion of a patient's body, comprising:
an inflatable component to be positioned between a body portion and an underlying surface, the inflatable component comprising:
one or more inflatable cells, and
a dressing, attached to the one or more inflatable cells, to be secured to a patient's skin;
tubing coupled to the one or more inflatable cells; and
a pump, coupled to the tubing, to inflate the one or more inflatable cells.

47. The system of claim 46, wherein the pump is configured to alternately inflate and deflate the one or more inflatable cells.

48. The system of claim 46, wherein the pump is integrated with a negative-pressure wound therapy system.

49. A method of providing pressure relief for a body part, comprising:
securing an inflatable device to a body portion of a patient, the inflatable device comprising one or more inflatable cells and a dressing attached to the one or more inflatable cells, the securing including placing the dressing on the patient's skin;
coupling the inflatable device to a pump; and
repeatedly inflating and deflating the inflatable device while secured to the body portion.

50. An inflatable device to be positioned between a body portion and an underlying surface, comprising:
one or more inflatable cells; and
an adhesive to secure the one or more inflatable cells to a patient's skin.

51. An inflatable device to provide pressure relief to a body part, comprising:
one or more inflatable cells;
a bottom elastic surface to be positioned against an underlying support and configured to expand when the one or more inflatable cells are inflated and to contract when the one or more inflatable cells are deflated; and
a top inelastic surface to be secured to a portion of a patient's body.

52. The inflatable device of claim 51, when the inelastic surface comprises an adhesive to attach the inelastic surface to the patient.

53. The inflatable device of claim 51, when the elastic surface is configured to flatten smoothly against the underlying support when the one or more inflatable cells are deflated.

54. The inflatable device of claim 51, wherein the one or more inflatable cells include first and second independently inflatable cells.

55. The inflatable device of claim 51, when the inelastic surface comprises a dressing.

56. A method of providing pressure relief to a body part, comprising:
securing an inflatable device to a body portion of a patient, the inflatable device comprising one or more inflatable cells, a bottom elastic surface, and a top inelastic surface, the securing including positioning the top inelastic surface under the body portion;
coupling the inflatable device to a pump; and
repeatedly inflating and deflating the inflatable device while the top inelastic surface remains positioned under the body portion.

57. The method of claim 56, wherein the one or more inflatable cells include first and second independently inflatable cells, the method further comprising:
inflating the first inflatable cell when the second inflatable cell is deflated, to tilt the patient.
